(19) United States
(12) Patent Application Publication
(10) Pub. No.: US 2021/0307729 A1
(43) Pub. Date: Oct. 7, 2021

(54) FECAL SAMPLER

(71) Applicant: ProciseDx Inc., San Diego, CA (US)

(72) Inventors: David Booker, San Diego, CA (US); Robert Eisele, Carlsbad, CA (US)

(73) Assignee: ProciseDx Inc., San Diego, CA (US)

(21) Appl. No.: 17/240,517

(22) Filed: Apr. 26, 2021

Related U.S. Application Data
(63) Continuation of application No. PCT/IB2019/059378, filed on Oct. 31, 2019.
(60) Provisional application No. 62/760,800, filed on Nov. 13, 2018.

(51) Int. Cl. A61B 10/00; B01L 3/00

(52) U.S. Cl. CPC A61B 10/0038; B01L 3/50825

(57) ABSTRACT

Provided herein are containers for holding a portion of a biological sample such as a fecal sample. The containers include a body that can be sealed with an upper cap that is attached to a sampling pin and a grip member. The sampling pin is configured to collect a predetermined amount of sample in one or more sampling grooves, with excess sample amount optionally removed from the sampling pin by an interior flange of the container body. The grip member is configured to provide the user of the container with improved torque and grip, and to distance the fingers of the user from the sample being collected. Also provided are kits and methods including the disclosed containers.

### CROSS-REFERENCE TO RELATED APPLICATIONS

This application is a continuation of International Patent Application No. PCT/IB2019/059378, filed Oct. 31, 2019, which claims priority to US Provisional Application No: 62/760,800, filed Nov. 13, 2018, the teachings of which are hereby incorporated by reference in their entirety for all purposes.

### BACKGROUND

Stool tests provide medical practitioners with an important diagnostic tool for the identification and characterization of various gastrointestinal disorders. These tests, which involve the collection and analysis of fecal matter from patients, examine the composition and chemistry of stool samples and correlate results with those of previously determined observations and benchmarks. For example, fecal occult blood tests can be used to diagnose colorectal cancer, stomach cancer, or other conditions that cause bleeding in the gastrointestinal system. Parasitic diseases such as ascariasis, hookworm, strongyloidiasis, and whipworm; infections from the bacteria such as Clostridium difficile; and the presence of viruses such as rotavirus can each be identified with various microscopy or other microbiology tests. Fecal pH tests and fecal fat tests can also be used to determine conditions such as lactose intolerance and fat malabsorption, respectively.

To provide material for such tests, devices are often used to collect and store fecal samples in amounts needed for different diagnostic assays. These collection devices are typically supplied to patients by their health care providers, and the patients often must then use the devices at home to acquire the sample. Because the sample collection is thus commonly performed by individuals who are not medical professionals, the need exists for improvements to the collection devices that can increase compliance and ease-of-use when operated by members of the general public. The present disclosure provides these and other needs.

### BRIEF SUMMARY

In one aspect, the disclosure is to a container for holding a biological sample. The container comprises a body having an upper end and a lower end. The container further comprises an upper cap and a lower cap. The upper cap comprises an interior surface and an exterior surface, and is configured to removably seal the upper end of the container. The lower cap is configured to removably seal the lower end of the container. The container further comprises a sampling pin having a proximal end, a distal end, and at least one groove proximate to the distal end. The proximal end of the sampling pin is connected to and extends from the interior surface of the upper cap. The container further comprises a grip member connected to the exterior surface of the upper cap and extending substantially opposite the sampling pin. The grip member comprises a Y shape which includes a shaft and two prongs.

In some embodiments, the body further comprises an internal flange dividing the body into an upper portion and a lower portion. The internal flange can comprises a funnel shape having a narrowing cross-sectional area in the direction from the upper portion of the body to the lower portion of the body. The internal flange can be configured to allow insertion of the sampling pin distal end therethrough from the upper portion of the body to the lower portion of the body. The upper cap can lock with the body upon insertion of a sufficient portion of the upper end of the body into the upper cap. The upper end of the body can comprise a lock groove, and the interior surface of the upper cap can comprise a lock ridge substantially complementary to the lock groove. The lock groove can span a majority of the circumference of the body, and the lock ridge can span a majority of the circumference of the upper cap inner surface.

In some embodiments, the ratio of the axial length of the grip member to the axial length of the upper cap is between 0.2 and 5.0. In some embodiments, at least one of the shaft and the two prongs comprises at least one substantially planar side. The at least one substantially planar side can comprise surface modifications configured to improve friction upon handling of the grip member. The surface modifications can comprise one or more grip ridges. In some embodiments, the ratios of the axial lengths of each prong to the axial length of the shaft are each independently between 0.2 and 5. In some embodiments, each prong forms an angle with the shaft, wherein each angle is independently between 120 degrees and 180 degrees.

In another aspect the disclosure is to a method for storing a portion of a biological sample. The method provides providing a container comprising a body, an upper cap, a sampling pin, a grip member, a lower cap, and a liquid within the body. In some embodiments, the liquid comprises a buffer. In some embodiments, the biological sample comprises a fecal sample. The body comprises an upper end and a lower end. The upper cap comprises an interior surface and an exterior surface, and is configured to removably seal the upper end of the body. The sampling pin comprises a proximal end, a distal end, and at least one sampling groove proximate to the distal end. The proximal end of the sampling pin is connected to and extends from the interior surface of the upper cap. The grip member is connected to the exterior surface of the upper cap, and extends substantially opposite the sampling pin. The grip member also comprises a Y shape which includes a shaft and two prongs. The lower cap is configured to removably seal the lower end of the body. The method further comprises inserting the sampling pin distal end into the biological sample such that the at least one sampling groove is within the biological sample. The method further comprises removing the sampling pin from the biological sample, thereby collecting a portion of the biological sample within the least one sampling groove. The method further comprises sealing the upper end of the body with the upper cap, thereby submerging the sampling pin distal end in the liquid, and thereby storing the portion of the biological sample.

In some embodiments, the body further comprises an internal flange dividing the body into an upper portion and a lower portion, wherein the liquid is within the lower portion, and wherein the sealing comprises passing the sampling pin distal end through the internal flange, thereby partitioning any excess amount of the portion of the biological sample within the upper portion. In some embodiments, the internal flange comprises a funnel shape having a narrowing cross-sectional area in the direction from the upper portion of the body to the lower portion of the body. In some embodiments the upper cap locks with the body upon insertion of a sufficient portion of the upper end of the body into the upper cap. The upper end of the body can comprise a lock groove, and the interior surface of the upper cap can comprise a lock ridge substantially complementary to the lock groove. The lock groove can span a majority of the circumference of the body, and the lock ridge can span a majority of the circumference of the upper cap inner surface.

In some embodiments, the inserting, removing, and sealing comprise holding the grip member. In some embodiments, the ratio of the axial length of the grip member to the axial length of the upper cap is between 0.2 and 5. In some embodiments, at least one of the shaft and the two prongs comprises at least one substantially planar side. The at least one substantially planar side can comprise surface modifications configured to improve friction upon holding the grip member. In some embodiments, the ratios of the axial lengths of each prong to the axial length of the shaft are each independently between 0.2 and 5. In some embodiments, each prong forms an angle with the shaft, wherein each angle is independently between 120 degrees and 180 degrees.

In another aspect, the disclosure is to a kit comprising any of the containers disclosed herein, wherein the container is an inner container. The kit further comprises an outer container comprising an opening configured to allow insertion and removal of the inner container within the outer container. The kit further comprises an outer container cap configured to removably seal the opening. In some embodiments, the kit further comprises an absorbent sleeve positioned between the inner container and the outer container.

### BRIEF DESCRIPTION OF THE DRAWINGS

FIG. 1A is an illustration of a container in accordance with an embodiment.

FIG. 1B is an illustration of a transparent view of the container of FIG. 1.

FIG. 2 is an illustration of the body of the container of FIGS. 1A and 1B

FIG. 3 is an illustration of the upper cap, sampling pin, and grip member of the container of FIGS. 1A and 1B.

FIG. 4 is an illustration of a kit in accordance with an embodiment.

FIG. 5 is a flowchart of a process in accordance with an embodiment.

### DETAILED DESCRIPTION

The present disclosure generally relates to containers for holding a portion of a biological sample, such as a fecal sample. These containers provide advantageous properties allowing them to be used more easily and reliably by individuals in the process of collecting a stool sample for medical testing. For example, it can be beneficial for fecal sample containers to be easily manipulated by differently abled individuals, such as elderly subjects who may have reduced grip strength and precision. It can also be of benefit for the individuals to reduce their potential contact with the fecal sample during the sampling. It has been difficult, however, to achieve such results with existing stool collection devices.

Most conventional fecal collection devices include a rod, stick, or scoop that must be used to pick up material from a stool sample for addition into a container. This rod, stick, or scoop is typically an element of the container lid, so that delivering the sample to the container has the additional effect of closing the container. The container lids are usually of a screw-on or plug variety, in which a user must tightly grasp the bulky lid to remove and insert it into the container, and to scrape or scoop the fecal material to be collected. Moreover, the separation between the fingers of the user and the fecal matter being sampled is often small in these conventional devices. This can increase the likelihood that that user will inadvertently contact the stool with his or her fingers, and can reduce compliance with the sample collection procedures.

The inventors have now discovered that specific container designs permit an increase in grip and torque during collection and storing of a portion of a biological sample such as a fecal sample. In particular, the provided containers include a grip member attached to and extending from the lid. Importantly, the particular dimensions and geometries of the grip member have been found to provide easier manipulation and handling of the container cap and its associated sampling pin. The grip member also provides the additional advantage of increasing the distance between the collected fecal sample and the fingers of an operator holding the grip member of the improved design so as to not contaminate the sample.

### I. Containers

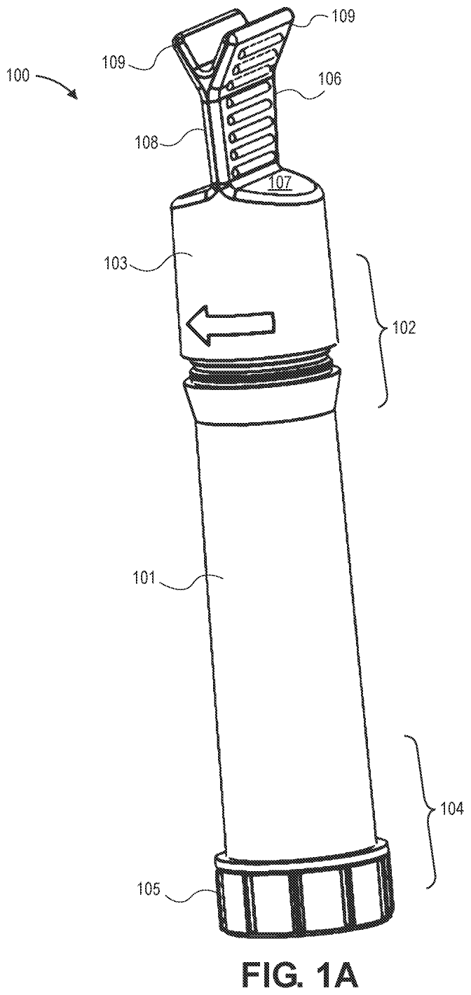

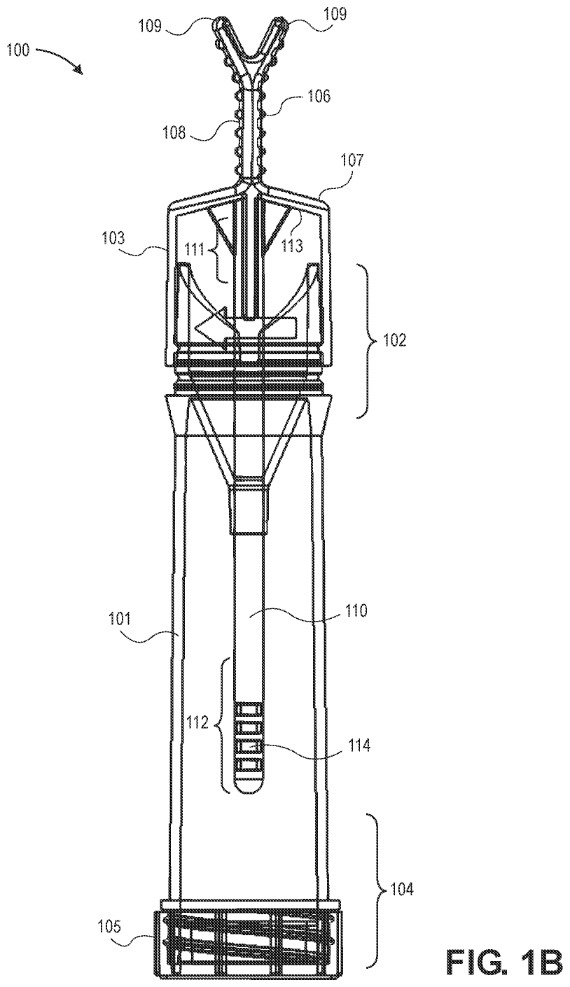

FIGS. 1A and 1B illustrate a container for holding a biological sample in accordance with an embodiment. The side view in FIG. 1A is of the container (100) in a closed configuration, and the alternate side view in FIG. 1B is a transparent representation of the same container (100). The container (100) includes a body (101), which is sealed at its upper end (102) by an upper cap (103), and at its lower end (104) by a lower cap (105). A grip member (106) is connected to the exterior surface (107) of the upper cap (103), extending from the upper cap (103) in a direction substantially away from, or opposite to, the body (101). The grip member has a Y shape which includes a shaft (108) and two prongs (109).

As can be seen in the transparent view of FIG. 1B, the container also includes a sampling pin (110) having a proximal end (111) and a distal end (112). The proximal end (111) is connected to the interior surface (113) of the upper cap (103), and extends from the upper cap (103) in a direction substantially away from, or opposite to, the grip member (106). The sampling pin (110) also includes at least one sampling groove (114) proximate to the distal end (112). There may be one of more (1, 2, 3, 4, 5, 6, or more) sampling groove(s) (114) to meter or measure the amount of sample.
A. Container Body and Lower Cap

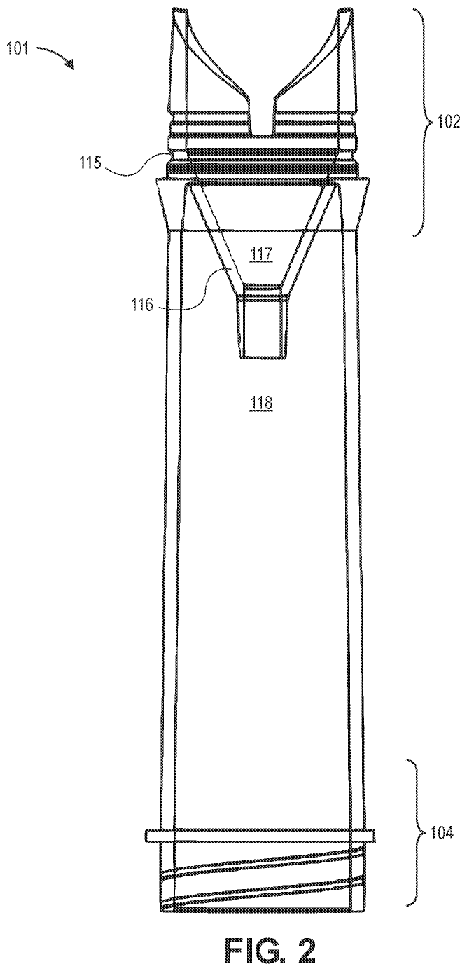

FIG. 2 provides a cross-sectional side view of the body (101) of the container (100) of FIGS. 1A and 1B. In some embodiments, and as shown in FIG. 2, the body (101) has a primarily cylindrical shape. It should be appreciated that the body (101) can have an alternative shape, such as for example, rectangular, as long as the body (101) encloses a hollow interior chamber when sealed at the upper end (102) by the upper cap, and at the lower end (104) by the lower cap. In some embodiments, the upper end (102) and the lower end (104) can each have threading or grooves that are substantially complementary to features of the upper cap and the lower cap, such that these upper and lower cap features mate with the threading or grooves of the body (101) when used to create removable seals. In some embodiments, the lower cap is configured to attach to the body (101) by screwing onto the lower end (104). In some embodiments, the lower cap is configured to attach to the body (101) by pressing or snapping onto the lower end (104).

In certain aspects, the upper end (102) of the body (101) includes one or more lock grooves (115) that are substantially complementary to lock ridges of the upper cap. The body (101) can include one lock groove (115), or two or more lock grooves (115). The one or more lock grooves (115) can span a minor fraction of the outer surface of the body, or can span a majority of the outer surface (e.g., a majority of the circumference) of the body. In some embodiments, and as shown in FIG. 2, the lock groove (115) spans the entire outer circumference of the body. The positioning of the one or more lock grooves (115) at the upper end (102) of the body (101) can be such that when a portion of the upper end (102) above the lock grooves (115) is inserted into the upper cap, the upper cap seals the upper end (102) of the body (101) in a removable fashion. If instead a sufficient portion of the upper end (102), e.g., a portion including the lock grooves (115), is inserted into the upper cap, then the upper cap seals the upper end (102) in a locked fashion in which the upper cap is not easily removable.

The container body can have an average outer diameter that is, for example, between 15 mm and 60 mm, e.g., between 15 mm and 42 mm, between 19.5 mm and 46.5 mm, between 24 mm and 51 mm, between 28.5 mm and 55.5 mm, or between 33 mm and 60 mm. In terms of upper limits, the body outer diameter can be less than 60 mm, e.g., less than 55.5 mm, less than 51 mm, less than 46.5 mm, less than 42 mm, less than 37.5 mm, less than 33 mm, less than 28.5 mm, less than 24 mm, or less than 19.5 mm. In terms of lower limits, the body outer diameter can be greater than 15 mm, e.g., greater than 19.5 mm, greater than 24 mm, greater than 28.5 mm, greater than 33 mm, greater than 37.5 mm, greater than 42 mm, greater than 46.5 mm, greater than 51 mm, or greater than 55.5 mm. Larger diameters, e.g., greater than 60 mm, and smaller diameters, e.g., less than 15 mm, are also contemplated.

The container body can have a length that is, for example, between 30 mm and 200 mm, e.g., between 30 mm and 132 mm, between 47 mm and 149 mm, between 64 mm and 166 mm, between 81 mm and 183 mm, or between 98 mm and 200 mm. In terms of upper limits, the body length can be less than 200 mm, e.g., less than 183 mm, less than 166 mm, less than 149 mm, less than 132 mm, less than 115 mm, less than 98 mm, less than 81 mm, less than 64 mm, or less than 47 mm. In terms of lower limits, the body length can be greater than 30 mm, e.g., greater than 47 mm, greater than 64 mm, greater than 81 mm, greater than 98 mm, greater than 115 mm, greater than 132 mm, greater than 149 mm, greater than 166 mm, or greater than 183 mm. Larger lengths, e.g., greater than 200 mm, and smaller lengths, e.g., less than 30 mm, are also contemplated.

The ratio of the body length to the body diameter can be, for example, between 2 and 10, e.g., between 2 and 6.8, between 2.8 and 7.6, between 3.6 and 8.4, between 4.4 and 9.2, or between 5.2 and 10. In terms of upper limits, the ratio of the body length to the body diameter can be less than 10, e.g., less than 9.2, less than 8.4, less than 7.6, less than 6.8, less than 6, less than 5.2, less than 4.4, less than 3.6, or less than 2.8. In terms of lower limits, the ratio of the body length to the body diameter can be greater than 2, e.g., greater than 2.8, greater than 3.6, greater than 4.4, greater than 5.2, greater than 6, greater than 6.8, greater than 7.6, greater than 8.4, or greater than 9.2. Higher ratios, e.g., greater than 10, and lower ratios, e.g., less than 2, are also contemplated.

In some embodiments, the body includes an internal flange (116) that divides the body into an upper portion (117) and a lower portion (118). In certain aspects, and as is shown in FIG. 2, the body includes an internal flange (116) having a funnel shape with a narrowing cross-sectional area in the direction from the upper portion (117) to the lower portion (118). The opening of the internal flange (116) at the upper portion can have an inner diameter configured to be large enough to allow the distal end of the sampling pin to be inserted therethrough from the upper portion (117) to the lower portion (118). The inner diameter of the internal flange (116) opening can also be configured to be small enough to prevent material that is attached to the sampling pin, and not within the sampling groove, from entering the lower portion (118) of the body (101). In this way, the design of the internal flange (116), in combination with the design of the sampling pin and its sampling groove(s), can effectively control the amount of solid sample material added to the lower portion (118) of the body. In other words, a precise amount of sample is delivered into the lower portion of the body. In certain instances, the amount of sample is proportional to the number of groove(s).

The inner diameter of the internal flange can be, for example, between 1 mm and 10 mm, e.g., between 1 mm and 6.4 mm, between 1.9 mm and 7.3 mm, between 2.8 mm and 8.2 mm, between 3.7 mm and 9.1 mm, or between 4.6 mm and 10 mm. In terms of upper limits, the internal flange inner diameter can be less than 10 mm, e.g., less than 9.1 mm, less than 8.2 mm, less than 7.3 mm, less than 6.4 mm, less than 5.5 mm, less than 4.6 mm, less than 3.7 mm, less than 2.8 mm, or less than 1.9 mm. In terms of lower limits, the internal flange diameter can be greater than 1 mm, e.g., greater than 1.9 mm, greater than 2.8 mm, greater than 3.7 mm, greater than 4.6 mm, greater than 5.5 mm, greater than 6.4 mm, greater than 7.3 mm, greater than 8.2 mm, or greater than 9.1 mm. Larger diameters, e.g., greater than 10 mm, and smaller diameters, e.g., less than 1 mm, are also contemplated.

In some embodiments, the container includes a liquid within the body. The liquid can be in the lower portion of the body (the reservoir) for those embodiments that include an internal flange. In some embodiments, the liquid is an aqueous solution. In some embodiments, the liquid includes one or more salts, one or more buffers, one or more assay reagents, or a combination thereof. In some embodiments, the liquid is an aqueous buffer solution suitable for stabilizing and storing a portion of a biological sample upon insertion therein. For example, the liquid can have a pH and/or an osmolarity suitable for stabilizing and storing the sample portion.
B. Upper Cap, Sampling Pin, and Grip Member

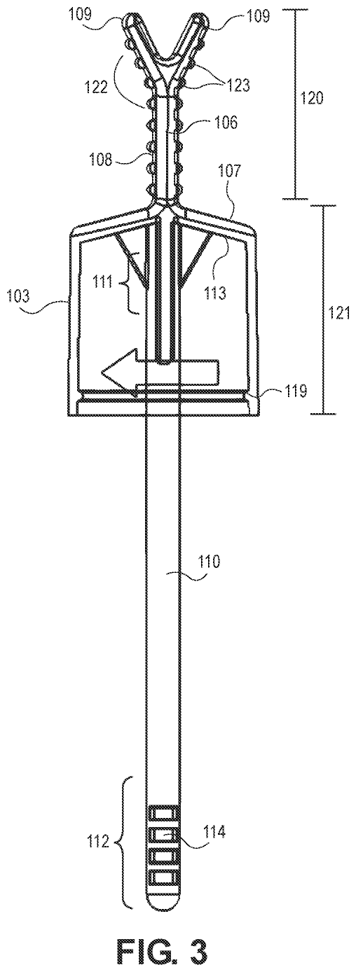

FIG. 3 provides a cross-sectional side view of the upper cap (103), sampling pin (110), and grip member (106) of the container (100) of FIGS. 1A and 1B. The upper cap (103) generally has an interior shape that is substantially complementary to that of the upper end of the container body, such that the upper cap (103) and the upper end of the body can mate to form a removable seal. In some embodiments, the upper cap (103) is configured to attach to the body by screwing onto the upper end. In some embodiments, the upper cap (103) is configured to attach to the body by pressing or snapping onto the upper end.

In certain aspects, the interior surface (113) of the upper cap (103) includes one or more lock ridges (119) that are substantially complementary to lock grooves of the upper end of the container body. The upper cap (103) can include one lock ridge (119), or two or more lock ridges (119). The one or more lock ridges (119) can span a minor fraction of the interior surface (113) of the upper cap (103), or can span a majority of the interior surface (e.g., a majority of the circumference) of the upper cap (103). In some embodiments, and as shown in FIG. 3, the lock ridge (119) spans the entire inner circumference of the upper cap (103). The positioning of the one or more lock ridges (119) on the interior surface (113) of the upper cap (113) can be such that when a portion of the body above the lock grooves is inserted into the upper cap (103), the upper cap (103) seals the upper end of the body in a removable fashion. If instead a sufficient portion of the body, e.g., a portion including the lock grooves, is inserted into the upper cap, then the lock grooves mate with the lock ridges (119), and the upper cap (103) seals the upper end of the body in a locked fashion in which the upper cap (103) is not easily removable. In this manner, a chain-of-custody of the sample is maintained.

The proximal end (111) of the sampling pin (110) is attached to and extends from the interior surface (113) of the upper cap (103). Generally, the sampling pin is centrally located within the upper cap (103), and is substantially straight, such that upon insertion of the upper end of the body into the upper cap, the sampling pin passes through the hole of an internal flange of the body, if present. The distal end (112) of the sampling pin (110) includes one or more sampling grooves (114). The sampling pin can include one sampling groove (114), or two or more sampling grooves (114). The one or more sampling grooves (114) can span a minor fraction of the outer surface of the sampling pin distal end (112), or can span a majority of the outer surface of the distal end (112). The one or more sampling grooves can be oriented in a generally axial direction along the sampling pin, or can, as show in FIG. 3, be oriented in a generally tangential direction. In some embodiments, the sampling grooves span the entire outer circumference of the sampling pin. The sampling grooves can be on one side of the distal end of the sampling pin. The sampling grooves can be substantially mirrored on two opposite sides of the distal end of the sampling pin.

The dimensions of the one or more sampling grooves can be configured such that the combined interior volumes of the grooves corresponds with a targeted amount of the biological sample to be collected. In this way, the design of the sampling grooves and the sampling pin, in combination with the design of the internal flange of the container body, can effectively control the amount of solid sample added to the lower portion of the body. The total volume defined by the sampling grooves can be, for example, between 10 μL and 1000 μL, e.g., between 10 μL and 160 μL, between 16 μL and 250 μL, between 25 μL and 400 μL, between 40 μL and 630 μL, or between 63 μL and 1000 μL. In terms of upper limits, the total sampling groove volume can be less than 1000 μL, e.g., less than 630 μL, less than 400 μL, less than 250 μL, less than 160 μL, less than 100 μL, less than 63 μL, less than 40 μL, less than 25 μL, or less than 16 μL. In terms of lower limits, the total sampling groove volume can be greater than 10 μL, e.g., greater than 16 μL, greater than 25 μL, greater than 40 μL, greater than 63 μL, greater than 100 μL, greater than 250 μL, greater than 400 μL, or greater than 630 μL. Larger volumes, e.g., greater than 1000 μL, and smaller volumes, e.g., less than 10 μL, are also contemplated. As the volume of liquid is known and the amount of sample is known, a concentration of solution can thus be determined.

In some embodiments, the sampling pin has a primarily cylindrical shape. It should be appreciated that the sampling pin can have an alternate shape, such as, for example, rectangular. The length of the sampling pin can be configured such that the sampling pin is long enough to sufficiently extend through a container body internal flange, when present, and short enough to permit the upper cap to close and seal the body. The sampling pin can have a length that is, for example, between 10 mm and 200 mm, e.g., between 10 mm and 124 mm, between 29 mm and 143 mm, between 48 mm and 162 mm, between 67 mm and 181 mm, or between 86 mm and 200 mm. In terms of upper limits, the sampling pin length can be less than 200 mm, e.g., less than 181 mm, less than 162 mm, less than 143 mm, less than 124 mm, less than 105 mm, less than 86 mm, less than 67 mm, less than 48 mm, or less than 29 mm. In terms of lower limits, the sampling pin length can be greater than 10 mm, e.g., greater than 29 mm, greater than 48 mm, greater than 67 mm, greater than 86 mm, greater than 105 mm, greater than 124 mm, greater than 143 mm, greater than 162 mm, or greater than 181 mm. Larger lengths, e.g., greater than 200 mm, and smaller lengths, e.g., less than 10 mm, are also contemplated.

The outer diameter of the sampling pin can be configured such that the sampling pin is narrow enough to permit the distal end of the sampling pin to be inserted through the opening of a container body internal flange, when present. The sampling pin outer diameter can be configured such that the sampling pin is wide enough to prevent material that is attached to the sampling pin, and not within the one or more sampling grooves, from entering the lower portion of the body upon passing the sampling pin distal end through the internal flange opening. The sampling pin outer diameter can be, for example, between 1 mm and 10 mm, e.g., between 1 mm and 6.4 mm, between 1.9 mm and 7.3 mm, between 2.8 mm and 8.2 mm, between 3.7 mm and 9.1 mm, or between 4.6 mm and 10 mm. In terms of upper limits, the sampling pin outer diameter can be less than 10 mm, e.g., less than 9.1 mm, less than 8.2 mm, less than 7.3 mm, less than 6.4 mm, less than 5.5 mm, less than 4.6 mm, less than 3.7 mm, less than 2.8 mm, or less than 1.9 mm. In terms of lower limits, the sampling pin diameter can be greater than 1 mm, e.g., greater than 1.9 mm, greater than 2.8 mm, greater than 3.7 mm, greater than 4.6 mm, greater than 5.5 mm, greater than 6.4 mm, greater than 7.3 mm, greater than 8.2 mm, or greater than 9.1 mm. Larger diameters, e.g., greater than 10 mm, and smaller diameters, e.g., less than 1 mm, are also contemplated.

The ratio of the sampling pin length to the sampling pin diameter can be, for example, between 5 and 50, e.g., between 5 and 32, between 9.5 and 36.5, between 14 and 41, between 18.5 and 45.5, or between 23 and 50. In terms of upper limits, the ratio of the sampling pin length to the sampling pin diameter can be less than 50, e.g., less than 45.5, less than 41, less than 36.5, less than 32, less than 27.5, less than 23, less than 18.5, less than 14, or less than 9.5. In terms of lower limits, the ratio of the sampling pin length to the sampling pin diameter can be greater than 5, e.g., greater than 9.5, greater than 14, greater than 18.5, greater than 23, greater than 27.5, greater than 32, greater than 36.5, greater than 41, or greater than 45.5. Higher ratios, e.g., greater than 50, and lower ratios, e.g., less than 5, are also contemplated.

The grip member (106) is attached to and extends from the exterior surface (107) of the upper cap (103). Generally, the grip member (106) is centrally located on the upper cap (103), and extends directly opposite the sampling pin (110). The shaft (108) of the Y-shaped grip member (106) can have an axial length extending from the upper cap (103) to the grip member prongs (109) that is, for example, between 5 mm and 50 mm, e.g., between 5 mm and 32 mm, between 9.5 mm and 36.5 mm, between 14 mm and 41 mm, between 18.5 mm and 45.5 mm, or between 23 mm and 50 mm. In terms of upper limits, the axial length of the grip member shaft (108) can be less than 50 mm, e.g., less than 45.5 mm, less than 41 mm, less than 36.5 mm, less than 32 mm, less than 27.5 mm, less than 23 mm, less than 18.5 mm, less than 14 mm, or less than 9.5 mm. In terms of lower limits, the axial length of the grip member shaft (108) can be greater than 5 mm, e.g., greater than 9.5 mm, greater than 14 mm, greater than 18.5 mm, greater than 23 mm, greater than 27.5 mm, greater than 32 mm, greater than 36.5 mm, greater than 41 mm, or greater than 45.5 mm. Larger lengths, e.g., greater than 50 mm, and smaller lengths, e.g., less than 5 mm, are also contemplated.

Each prong (109) of the Y-shaped grip member (106) can independently have an axial length extending from the grip member shaft (108) that is, for example, between 5 mm and 50 mm, e.g., between 5 mm and 32 mm, between 9.5 mm and 36.5 mm, between 14 mm and 41 mm, between 18.5 mm and 45.5 mm, or between 23 mm and 50 mm. In terms of upper limits, the axial length of each grip member prong (109) can independently be less than 50 mm, e.g., less than 45.5 mm, less than 41 mm, less than 36.5 mm, less than 32 mm, less than 27.5 mm, less than 23 mm, less than 18.5 mm, less than 14 mm, or less than 9.5 mm. In terms of lower limits, the axial length of each grip member prong (109) can independently be greater than 5 mm, e.g., greater than 9.5 mm, greater than 14 mm, greater than 18.5 mm, greater than 23 mm, greater than 27.5 mm, greater than 32 mm, greater than 36.5 mm, greater than 41 mm, or greater than 45.5 mm. Larger lengths, e.g., greater than 50 mm, and smaller lengths, e.g., less than 5 mm, are also contemplated.

The ratio of the axial length of the shaft to the average axial length of the prongs can be, for example, between 0.2 and 5, e.g., between 0.2 and 3.08, between 0.68 and 3.56, between 1.16 and 4.04, between 1.64 and 4.52, or between 2.12 and 5. In terms of upper limits, the ratio of the shaft axial length to the average prong axial length can be less than 5, e.g., less than 4.52, less than 4.04, less than 3.56, less than 3.08, less than 2.6, less than 2.12, less than 1.64, less than 1.16, or less than 0.68. In terms of lower limits, the ratio of the shaft axial length to the average prong axial length can be greater than 0.2, e.g., greater than 0.68, greater than 1.16, greater than 1.64, greater than 2.12, greater than 2.6, greater than 3.08, greater than 3.56, greater than 4.04, or greater than 4.52. Higher ratios, e.g., greater than 5, and lower ratios, e.g., less than 0.2, are also contemplated.

The overall axial length (120) of the grip member (106) can be, for example, between 10 mm and 100 mm, e.g., between 10 mm and 64 mm, between 19 mm and 73 mm, between 28 mm and 82 mm, between 37 mm and 91 mm, or between 46 mm and 100 mm. In terms of upper limits, the grip member axial length can be less than 100 mm, e.g., less than 91 mm, less than 82 mm, less than 73 mm, less than 64 mm, less than 55 mm, less than 46 mm, less than 37 mm, less than 28 mm, or less than 19 mm. In terms of lower limits, the grip member axial length can be greater than 10 mm, e.g., greater than 19 mm, greater than 28 mm, greater than 37 mm, greater than 46 mm, greater than 64 mm, greater than 73 mm, greater than 82 mm, or greater than 91 mm. Larger lengths, e.g., greater than 100 mm, and smaller lengths, e.g., less than 10 mm, are also contemplated.

The ratio of the axial length (120) of the grip member (106) to the axial length (121) of the upper cap (103) can be, for example, between 0.2 and 5, e.g., between 0.2 and 3.08, between 0.68 and 3.56, between 1.16 and 4.04, between 1.64 and 4.52, or between 2.12 and 5. In terms of upper limits, the ratio of the grip member axial length to the upper cap axial length can be less than 5, e.g., less than 4.52, less than 4.04, less than 3.56, less than 3.08, less than 2.6, less than 2.12, less than 1.64, less than 1.16, or less than 0.68. In terms of lower limits, the ratio of the grip member axial length to the upper cap axial length can be greater than 0.2, e.g., greater than 0.68, greater than 1.16, greater than 1.64, greater than 2.12, greater than 2.6, greater than 3.08, greater than 3.56, greater than 4.04, or greater than 4.52. Higher ratios, e.g., greater than 5, and lower ratios, e.g., less than 0.2, are also contemplated.

Each prong (109) of the Y-shaped grip member (106) forms an angle (122) with the grip member shaft (108). In some embodiments, the two such angles (122) are identical to one another. In some embodiments, the two such angles (122) are different from one another. The angles (122) can each independently be, for example, between 120 degrees and 180 degrees, e.g., between 120 degrees and 156 degrees, between 126 degrees and 162 degrees, between 132 degrees and 168 degrees, between 138 degrees and 174 degrees, or between 144 degrees and 180 degrees. In terms of upper limits, each angle can be less than 180 degrees, e.g., less than 174 degrees, less than 168 degrees, less than 162 degrees, less than 156 degrees, less than 150 degrees, less than 144 degrees, less than 138 degrees, less than 132 degrees, or less than 126 degrees. In terms of lower limits, each angle can be greater than 120 degrees, e.g., greater than 126 degrees, greater than 132 degrees, greater than 138 degrees, greater than 144 degrees, greater than 150 degrees, greater than 156 degrees, greater than 162 degrees, greater than 168 degrees, or greater than 174 degrees. Smaller angles, e.g., less than 120 degrees, are also contemplated.

In certain aspects, at least one of the shaft and the two prongs of the grip member includes at least one substantially planar side. In some embodiments, and as shown in FIGS. 1A, 1B, 3, and 4, each of the shaft and the two prongs includes two substantially planar opposite sides. At least some of these planar sides can be facing outwards, such that they can be gripped by the fingers of a user manipulating the grip member. To improve this handling, at least one substantially planar side of the grip member can include surface modifications configured to increase friction between the grip member and the fingers of the user. Exemplary surface modifications include depressions such as grooves, protrusions such as ridges, and combinations thereof. In some embodiments, the surface modifications of the grip member includes one or more grip grooves. In some embodiments, the surface modifications of the grip member includes one or more grip ridges. In some embodiments, the surface modification of the grip member includes one or more grip grooves, and one or more grip ridges. In certain aspects, and as shown in FIG. 3, the shaft includes two surface-modified planar surfaces, and each prong includes one surface-modified planar surface, wherein the surface modifications are a series of parallel grip ridges (123).

### II. Kits

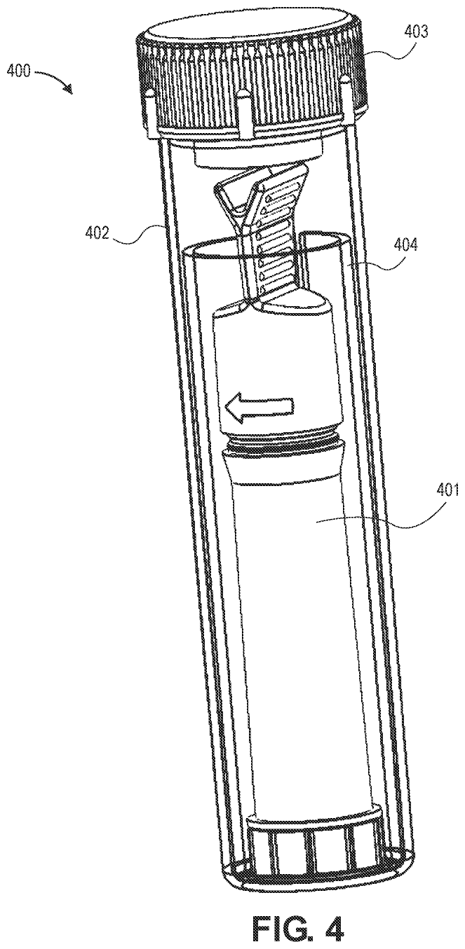

Also provided by this disclosure are kits for storing or transporting a container as disclosed herein. FIG. 4 illustrates an exemplary kit in accordance with an embodiment. The kit (400) includes an inner container (401) for holding a biological sample. The inner container (401) can be the container (100) of FIGS. 1A and 1B. The kit also includes an outer container (402) having an opening configured to allow insertion and removal of the inner container therein. The kit also includes an outer cap (403) configured to removably seal the opening to enclose the inner container (401) within the outer container (402). In some embodiments, the kit also includes an absorbent sleeve (404) positioned between the inner container (401) and the outer container (402). The kit can easily be packaged and mailed.

In some embodiments, the kit further includes a liquid within the body of the inner container. The liquid can be in the lower portion of the body for those embodiments that include an internal flange. In some embodiments, the liquid is an aqueous solution. In some embodiments, the liquid includes one or more salts, one or more buffers, one or more assay reagents, or a combination thereof. In some embodiments, the liquid is an aqueous buffer solution suitable for stabilizing and storing a portion of a biological sample upon insertion therein. For example, the liquid can have a pH and/or an osmolarity suitable for stabilizing and storing the sample portion.

In some embodiments, the kit further includes a portion of a biological sample. In some embodiments, the kit does not include a sample, and a sample is instead provided by a user of the kit. As used herein, “sample” can refer to any biological specimen or sample obtained from a subject. Samples include, without limitation, stool (i.e., feces), a tissue sample (e.g., tumor tissue) such as a biopsy of a tumor (e.g., needle biopsy) or a lymph node (e.g., sentinel lymph node biopsy), a tissue sample (e.g., tumor tissue) such as a surgical resection of a tumor, and cellular extracts thereof as well as internal intestinal mucosa. In some embodiments, the sample is a fecal sample. As used herein, “subject” refers to animals such as mammals, including, but not limited to, primates (e.g., humans), cows, sheep, goats, horses, dogs, cats, rabbits, rats, mice and the like. In certain embodiments, the subject is a human.

### III. Methods

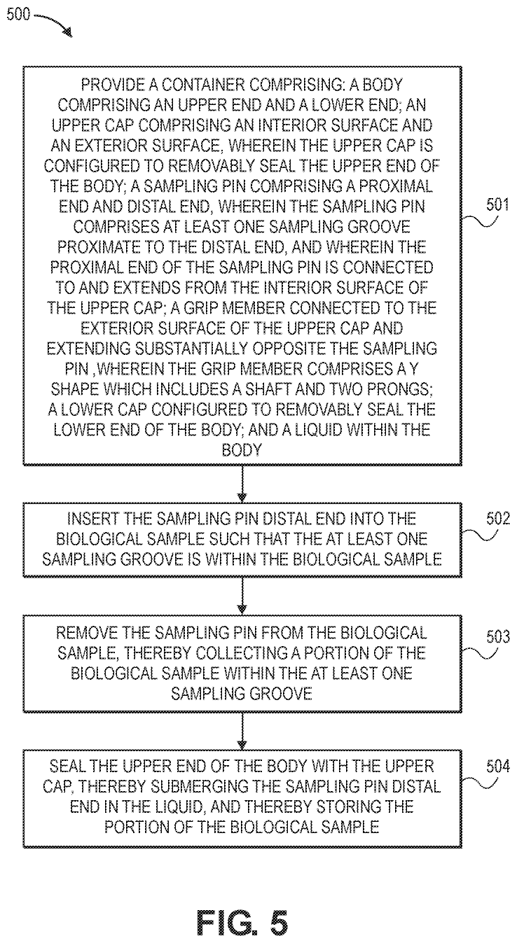

FIG. 5 presents a flowchart of a method (500) in accordance with an embodiment for storing a portion of a biological sample. In operation 501, a container is provided. The container includes: a body having an upper end and a lower end; an upper cap having an interior surface and an exterior surface, wherein the upper cap is configured to removably seal the upper end of the body; a sampling pin having a proximal end and distal end, wherein the sampling pin includes at least one sampling groove proximate to the distal end, and wherein the proximal end of the sampling pin is connected to and extends from the interior surface of the upper cap; a grip member connected to the exterior surface of the upper cap and extending substantially opposite the sampling pin ,wherein the grip member comprises a Y shape which includes a shaft and two prongs; a lower cap configured to removably seal the lower end of the body; and a liquid within the body. The container provided in the method can be any of the containers disclosed herein. In some embodiments, the liquid includes a buffer.

In operation 502, the sampling pin distal end is inserted into the biological sample such that at least one sampling groove is within the biological sample. In some embodiments, the biological sample is a fecal sample.

In operation 503, the sampling pin is removed from the biological sample, thereby collecting a portion of the biological sample within the at least one sampling groove. The at least one sampling groove can have an interior volume configured to have a size suitable for holding a predetermined amount of sample to be stored.

In operation 504, the upper end of the body is sealed with the upper cap, thereby submerging the sampling pin distal end in the liquid, and thereby storing the portion of the biological sample. In some embodiments, the body includes an internal flange as described above. In certain aspects, the sealing of the body with the upper cap includes passing the sampling pin distal end through the internal flange. In this way, the internal flange can act to remove any excess amount of the portion of the biological sample from the sampling pin, wherein the excess amount is that portion of the biological sample that is attached to the sampling pin and is not within the interior if the at least one sampling groove. The excess sample portion removed by the internal flange can thus be partitioned within the upper portion of the container body. The sample portion not removed by the internal flange corresponds to the predetermined amount of sample to be stored in the liquid of the lower portion of the container body.

Although the foregoing disclosure has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A container for holding a biological sample, the container comprising:
a body comprising an upper end and a lower end;
an upper cap comprising an interior surface and an exterior surface, wherein the upper cap is configured to removably seal the upper end of the body;
a sampling pin comprising a proximal end and distal end, wherein the sampling pin comprises at least one sampling groove proximate to the distal end, and wherein the proximal end of the sampling pin is connected to and extends from the interior surface of the upper cap;
a grip member connected to the exterior surface of the upper cap and extending substantially opposite the sampling pin, wherein the grip member comprises a Y shape which includes a shaft and two prongs; and
a lower cap configured to removably seal the lower end of the body.

2. The container of claim 1, wherein the body further comprises an internal flange dividing the body into an upper portion and a lower portion.

3. The container of claim 2, wherein the internal flange comprises a funnel shape having a narrowing cross-sectional area in the direction from the upper portion of the body to the lower portion of the body.

4. The container of claim 2, wherein the internal flange is configured to allow insertion of the sampling pin distal end therethrough from the upper portion of the body to the lower portion of the body.

5. The container of claim 4, wherein the upper cap locks with the body upon insertion of a sufficient portion of the upper end of the body into the upper cap.

6. The container of claim 5, wherein the upper end of the body comprises an lock groove, and wherein the interior surface of the upper cap comprises a lock ridge substantially complementary to the lock groove.

7. (canceled)

8. The container of claim 1, wherein the ratio of the axial length of the grip member to the axial length of the upper cap is between 0.2 and 5.

9. The container of claim 1, wherein at least one of the shaft and the two prongs comprises at least one substantially planar side, wherein the at least one substantially planar sides comprises surface modifications configured to improve friction upon handling of the grip member, and wherein the surface modifications comprise one or more grip ridges.

10-11. (canceled)

12. The container of claim 1, wherein the ratios of the axial lengths of each prong to the axial length of the shaft are each independently between 0.2 and 5.

13. The container of claim 1, wherein each prong forms an angle with the shaft, and wherein each angle is independently between 120 degrees and 180 degrees.

14. A method for storing a portion of a biological sample, the method comprising:
providing a container comprising:
a body comprising an upper end and a lower end;
an upper cap comprising an interior surface and an exterior surface, wherein the upper cap is configured to removably seal the upper end of the body;
a sampling pin comprising a proximal end and distal end, wherein the sampling pin comprises at least one sampling groove proximate to the distal end, and wherein the proximal end of the sampling pin is connected to and extends from the interior surface of the upper cap;
a grip member connected to the exterior surface of the upper cap and extending substantially opposite the sampling pin ,wherein the grip member comprises a Y shape which includes a shaft and two prongs;
a lower cap configured to removably seal the lower end of the body; and
a liquid within the body;
inserting the sampling pin distal end into the biological sample such that the at least one sampling groove is within the biological sample;
removing the sampling pin from the biological sample, thereby collecting a portion of the biological sample within the at least one sampling groove; and
sealing the upper end of the body with the upper cap, thereby submerging the sampling pin distal end in the liquid, and thereby storing the portion of the biological sample.

15. The method of claim 14, wherein the body further comprises an internal flange dividing the body into an upper portion and a lower portion, wherein the liquid is within the lower portion, and wherein the sealing comprises passing the sampling pin distal end through the internal flange, thereby partitioning any excess amount of the portion of the biological sample within the upper portion.

16. The method of claim 15, wherein the internal flange comprises a funnel shape having a narrowing cross-sectional area in the direction from the upper portion of the body to the lower portion of the body.

17. The method of claim 15, wherein the upper cap locks with the body upon insertion of a sufficient portion of the upper end of the body into the upper cap.

18. The method of claim 17, wherein the upper end of the body comprises a lock groove, and wherein the interior surface of the upper cap comprises a lock ridge substantially complementary to the lock groove.

19-20. (canceled)

21. The method of claim 14, wherein the ratio of the axial length of the grip member to the axial length of the upper cap is between 0.2 and 5.

22. The method of claim 14, wherein at least one of the shaft and the two prongs comprises at least one substantially planar side, wherein the at least one substantially planar side comprises surface modifications configured to improve friction upon holding the grip member, and wherein the surface modifications comprise one or more grip ridges.

23-24. (canceled)

25. The method of claim 14, wherein the ratios of the axial lengths of each prong to the axial length of the shaft are each independently between 0.2 and 5.

26. The method of claim 14, wherein each prong forms an angle with the shaft, wherein each angle is independently between 120 degrees and 180 degrees.

27-28. (canceled)

29. A kit comprising:
the container of claim 1, wherein the container is an inner container;
an outer container comprising an opening configured to allow insertion and removal of the inner container within the outer container;
an absorbent sleeve positioned between the inner container and the outer container; and
an outer container cap configured to removably seal the opening.

30. (canceled)
